(19) United States
(12) Patent Application Publication
(10) Pub. No.: US 2022/0068482 A1
(43) Pub. Date: Mar. 3, 2022

(54) INTERACTIVE TREATMENT PATHWAY INTERFACE FOR GUIDING DIAGNOSIS OR TREATMENT OF A MEDICAL CONDITION

(71) Applicant: Carnegie Mellon University, Pittsburgh, PA (US)

(72) Inventors: John Zimmerman, Pittsburgh, PA (US); Adam Perer, Pittsburgh, PA (US); Lisa C. Lohmueller, Pittsburgh, PA (US)

(21) Appl. No.: 17/435,431

(22) PCT Filed: Feb. 28, 2020
(86) PCT No.: PCT/US2020/020382

§ 371 Date: Sep. 1, 2021

Related U.S. Application Data
(60) Provisional application No. 62/919,212, filed on Mar. 1, 2019.

(51) Int. Cl. G16H 50/20; G16H 70/20; G16H 20/00; G16H 10/60

(52) U.S. Cl. CPC G16H 50/20; G16H 10/60; G16H 20/00; G16H 70/20

(57) ABSTRACT

Systems and methods for treating a patient for a medical condition using an interactive treatment pathway interface associated with the medical condition include receiving, through an interface, patient data representing a medical status of a patient. Treatment pathway data representing a sequence of actions for a particular treatment or diagnosis of a medical condition of the patient are retrieved. The interface guides the particular treatment or diagnosis of the medical condition by a traversal of the treatment pathway. A data trace is generated from additional patient data received during the traversal. The represents the instance of the particular treatment or diagnosis of the medical condition of the patient. The data are used to affect the particular treatment or diagnosis for the medical condition to the patient and inform machine learning operations for diagnosis or treatment of the medical condition.

### CLAIM OF PRIORITY

This application claims priority under 35 U.S.C. § 119(e) to U.S. Provisional Application Ser. No. 62/919,212, filed Mar. 1, 2019, the entire contents of which are hereby incorporated by reference.

### GOVERNMENT SUPPORT CLAUSE

This invention was made with government support under IIS-1723454 awarded by the National Science Foundation. The government has certain rights in the invention.

### TECHNICAL FIELD

This document describes an interface configured to provide guidance for treatment or diagnosis of a medical condition of a patient. More specifically, this document describes machine learning processes that assist a user in traversing a treatment pathway of a data structure to provide guidance, using an interface, for treatment of a medical condition of a patient.

### BACKGROUND

Computer systems can be used to transmit, receive, and/or process data. For instance, a server computer system can be used to receive and store resources (e.g., web content, such as a webpage), and make the content available to one or more client computer systems. Upon receiving a request for the content from a client computer system, the server computer system can retrieve the requested content, and transmit the content to the client computer system to fulfill the request.

Healthcare in the United States has been transitioning healthcare delivery away from paper-based record systems and towards systematic use of Electronic Health Records (EHR). Adoption of EHRs has been slower than expected, and many issues persist, such as problems with sharing information between different care providers and between different healthcare services. Adding to the challenges, researchers interested in the use of big data to advance healthcare have raised concerns about the quality of data in EHRs, noting that it is often missing, inaccurate, inconsistently entered, or diminished due to selective measurement. EHRs with low quality data significantly hamper the ability to bring computational intelligence into healthcare practice as poor quality data leads to poor quality inferences. Incomplete and inaccurate data also make data-driven innovation virtually impossible.

### SUMMARY

The system described in this document is configured for treating a patient for a medical condition using interactive clinical decision support tools. The clinical decision support tools provide guidance for treatment or diagnosis of a medical condition. Generally, the clinical decision support tools include a treatment pathway interface associated with the medical condition. Generally, the systems and processes described herein are configured to guide a user through a treatment or diagnosis process according to a treatment pathway. The treatment pathway represents a standardized or semi-standardized process for diagnosing a medical condition in a patient or treatment of a medical condition of a patient. This document describes an interface configured to guide the user (e.g., a physician or other medical service provider) to treat a patient using a treatment pathway. The interface assists a physician in choosing the correct treatment pathway using machine learning processes, subsequently described in detail. The interface ensures that each step of the treatment pathway is traversed during treatment and that data that should be provided to the system is collected before traversing the pathway to a subsequent step. The data processing system described herein uses the data collected during the guided traversal of the treatment pathway at each step to generate a data trace. The data trace represents how the treatment pathway was traversed. The data trace provides a standardized or semi-standardized data source that is compatible with one or more machine learning processes. The data processing system uses these data traces and various machine learning processes to suggest or effect a particular treatment or diagnose a particular medical condition for the patient.

Generally, EHRs contain both structured data and unstructured data. One of the largest sources of unstructured data are clinical notes. Clinical notes generally provide a descriptive narrative of a patient's interactions with their healthcare providers. Clinical notes capture the medical service provider's observations, impressions, plans, and actions during treatment of a patient. Clinical notes are generally an objective record of a patient's history and include physical findings and observations, medical reasoning, procedures and care provided to the patient, data to justify the care given, and clinical data for future research or data mining. Medical service providers generally attempt to document each point of interaction between patients and the healthcare system.

However, much of the authoring of clinical notes happens outside of a patient encounter, such as long after the patient has left the building. Medical service providers often struggle to find time to author their notes, creating them hours or even days after an encounter. This can result in the consequence in which clinical notes depend almost entirely on the recall of individual medical service providers. Furthermore, generating clinical notes manually can take a substantial amount of time. The effort to create them and to interact with EHRs has can cause physician burnout.

A challenge for generating effective clinical notes is that the notes must serve four distinct audiences with different needs. They include future medical service providers who will treat this patient, administrators who will perform billing, patients who now have access to their own online EHR, and computer systems that are increasingly being used behind the scenes to cull “big data” to improve healthcare. Compounding this complexity, clinical notes in many cases have become more focused on billing than on capturing an effective narrative describing a patient's health, with many EHR systems automatically add pages of billing-related content that clinicians must wade through when trying to understand the backstory of a new patient. This deviation from patient narrative to financial recordkeeping has been found to add to growing physician burnout.

When engaging with patients, medical service providers interact with more than just the EHR. They also interact with a number of different clinical decision support systems (CDSS). These systems are meant to support evidence based medicine by reminding clinicians of what needs to be done in specific situations (e.g., checklists, alerts) and by scaffolding their decision making when they work to diagnose and treat an individual patient (e.g., clinical guidelines, care pathways). CDSS generally improves the quality of healthcare; however, there is less support for the claim that CDSS make the delivery of healthcare more efficient or that they make clinicians feel more effective in their delivery of healthcare. Poorly designed CDS S can have negative impacts on efficiency of care delivery, and they can reduce the quality of care, likely due to clinicians choosing not to interact with a CDSS. The most common barrier listed that prevent clinicians from using a CDSS is the lack of time. Additionally, some clinicians have complained about the social performance of using a CDSS in front of a patient, making it appear the clinician needs help in order to do their job.

Clinical guidelines are a form of CDSS created from a synthesis of peer-reviewed research studies. They provide details that describe the most effective methods for diagnosis based on a set of common symptoms, and they provide details on how to treat patients once they have been diagnosed. These have been used for more than three decades to encourage the use of evidence based medicine, and repeated studies have shown their use improves compliance with standards of care and also improves patient outcomes. In addition, the healthcare industry has created international standards to describe their confidence in a set of guidelines (S1, S2, S3) where S3 guidelines are considered the most reliable.

Care Pathways (CPs) are one form of clinical guidelines. They detail steps for the care of patients with a specific clinical problem in a specific period of time. CPs often get presented as a flowchart, with specific measures used to determine which branch a clinician should follow when treating a specific patient. When used, CPs reduce variability in clinical practice, which improves the quality of patient care and use of healthcare resources. CPs also function as important tools for teaching and integrating new trainees or established clinicians into a department's workflow.

Determining a treatment pathway can be difficult because care pathways, and other clinical guidelines, have benefited from very few computational advances other than making them searchable via the web and making them context sensitive, so they transform for things like a patient's age or the location of care (e.g., primary care office, emergency department, intensive care unit). This is the same for other CDSS. All are just making the transition from paper to digital. Some of the challenges associated with CDSS adoption include a lack of consideration for clinicians' workflow and the role information systems currently play in work practice. In a review of clinical systems, researchers produced a ranked list of issues, and at the top of the list was the lack of interaction design.

However, a common barrier to use of CDSS by medical service providers is that current CDSS do not effectively collect data to support healthcare in adopting data-driven innovation (e.g., using machine learning approaches) for optimizing evidence-based medicine. In order to move healthcare a step closer to data-driven innovation, the data processing system described herein is configured to reduce the time and effort needed to engage with a CDSS by a medical service provider and log low-level trace data that allows medical service providers to conduct A/B tests and optimize the alignment of procedures and processes to desired outcomes.

The implementations described herein can provide various technical benefits for overcoming the challenges described above and improve data collection and the resulting treatment and diagnosis of medical conditions of patients. For instance, the techniques described herein enable diagnosis or treatment of the particular condition of the patient because the treatment of the patient using the treatment pathway is represented as standard or semi-standard data. The data trace can be input into a machine learning process or system, which can assist in recommending a diagnosis or treatment for a particular medical condition of the patient.

CDSS aid healthcare in ways that are similar to data driven innovation. CDSS enable medical service providers to provide services using the most optimal methods and procedures. When CDSS are used, they improve healthcare outcomes, most likely by improving consistency of care. The systems and method described herein allow CDSS to be integrated with the interface to enable medical service providers to generate data traces for effecting treatment of particular medical conditions and for diagnosing medical conditions An interactive CDSS used during an encounter with a patient could save clinicians time by semi-automating the creation of clinical notes. This can save clinicians time and motivate increased use of such a CDSS. An interactive CDSS also effectively logs low-level data. The interface enables the data processing system to measure and action sequences that provide data needed for A/B testing (e.g., using machine learning approaches), data for iterative improvement of the CDSS' decision support, and data needed to provide more personalized decision making for individual patients.

More specifically, the data processing system including the interface for an interactive CDSS provides several benefits. The data processing system increases clinician use of CDSS by saving medical service provider time currently spent writing clinical notes. The data processing system increases improves healthcare outcomes because it promotes increased compliance with the use of CDSS. This results in improves outcomes for effecting the treatment or diagnosis of a particular medical condition. The data processing system generates detailed log data (e.g., data trace) that allow for data driven innovation (e.g., using machine learning approaches). This data can be used to improve standards of care; to improve tools and processes used to deliver care, and to deliver high-quality, personalized healthcare.

Additional advantages include a consistent reduction in in-hospital patient complications, a reduction in length of stay, and a decrease in hospital costs. Studies on CPs used for specific diseases showed a decrease in the amount of therapy needed and a reduction in readmissions. Physicians indicated that the use of CPs improves multidisciplinary collaboration and quality of care. Implementation of CP systems have also been shown to improve patient communication and satisfaction. In other words, the data processing system allows users to interact with CDSS and generate semi-automated clinical notes, which provides insights on how a new kind of interactive system can collect detailed information on patients that can help scaffold more data-driven healthcare practice.

Generally the process for effecting, using the data trace, the particular treatment or diagnosis for the medical condition to the patient using the interface includes retrieving, from a data storage based on the patient data, treatment pathway data comprising a plurality of nodes, the plurality of nodes being linked to one another to form at least one treatment pathway through at least a portion of the plurality of nodes, the at least one treatment pathway representing a sequence of actions for a particular treatment or diagnosis of a medical condition of the patient. The treatment pathway can be selected for retrieval using a machine learning process, as subsequently described. The process generally includes representing, by the interface, a traversal through at least the portion of the nodes representing the at least one treatment pathway to guide the particular treatment or diagnosis of the medical condition. The traversal includes, for each node represented in the at least one treatment pathway, presenting, by the interface, at least one prompt for patient data or at least one suggested action for the particular treatment or diagnosis of the medical condition. The traversal includes receiving, in response to presenting the at least one prompt, additional patient data about the status of the patient. The traversal includes identifying, based on the additional patient data, either a subsequent node in the at least one treatment pathway or that the particular treatment or diagnosis is completed. Once the traversal is completed, the process includes generating, from the additional patient data received during the traversal, a data trace representing an instance of the particular treatment or diagnosis of the medical condition of the patient. The data processing system is configured to effect, using the data trace, the particular treatment or diagnosis for the medical condition to the patient. The data processing system stores the data trace representing the instance of the particular treatment or diagnosis in the data storage.

The systems and method described herein can include the features of one or more of the following embodiments. In a general aspect, a system is configured for treating a patient for a medical condition using an interactive treatment pathway interface associated with the medical condition by performing operations including receiving, through an interface, patient data representing a medical status of a patient. The operations include retrieving, from a data storage based on the patient data, treatment guidance data including a plurality of nodes, the plurality of nodes being linked to one another to form at least one treatment pathway through at least a portion of the plurality of nodes, the at least one treatment pathway representing a sequence of actions for a particular treatment or diagnosis of a medical condition of the patient. The operations include representing, by the interface, a traversal through at least the portion of the nodes representing the at least one treatment pathway to guide the particular treatment or diagnosis of the medical condition.

The traversal includes, for each node represented in the at least one treatment pathway: presenting, by the interface, at least one prompt for patient data or at least one suggested action for the particular treatment or diagnosis of the medical condition. The traversal includes receiving, in response to presenting the at least one prompt, additional patient data about the status of the patient. The traversal includes identifying, based on the additional patient data, either a subsequent node in the at least one treatment pathway or that the particular treatment or diagnosis is completed.

The operations include generating, from the additional patient data received during the traversal, a data trace representing an instance of the particular treatment or diagnosis of the medical condition of the patient. The operations include effecting, using the data trace, the particular treatment or diagnosis for the medical condition to the patient; and storing the data trace representing the instance of the particular treatment or diagnosis in the data storage.

In some implementations, the operations include presenting, to a user of the interface, a representation of the particular treatment or diagnosis, represented in the data trace. In some implementations, the user of the interface is a medical professional.

In some implementations, the operations include generating a classifier for selecting the treatment guidance data for retrieval from the data storage. Generally, the classifier is configured to: determine a probability value associated with each treatment pathway of the treatment guidance data, the probability value indicative of a likelihood of the treatment pathway being responsive to the medical status represented in the patient data. The classifier is used to select, for retrieving from the data storage, the treatment guidance data including the treatment pathway based on the probability value associated with that treatment pathway.

In some implementations, the classifier is generated using a machine learning process. Generating the classifier can include receiving training data including data traces for a plurality of patients, each data trace being associated with a particular treatment pathway. Generating the classifier can include training a classification function using the training data. The classification function is configured to determine the probability value associated with each treatment pathway of the treatment guidance data.

In some implementations, the operations include generating, in the data trace for each node of the treatment pathway, description data including a natural language description of the additional patient data associated with the node. In some implementations, the natural language description is generated using a machine learning process. In some implementations, the data trace comprises, for each node traversed, a time stamp representing when an action for treatment of the medical condition of the patient is performed.

In a general aspect, one or more non-transitory computer readable media are storing instructions that are executable by one or more processors configured to perform the foregoing operations and provide one or more of the foregoing features.

The details of one or more embodiments are set forth in the accompanying drawings and the description below. Other features and advantages will be apparent from the description and drawings, and from the claims.

### BRIEF DESCRIPTION OF THE DRAWINGS

FIG. 1 is a block diagram of an example computing environment for guiding diagnosis or treatment of a medical condition.

FIGS. 2A-2D illustrates an example of a treatment pathway.

FIG. 3 illustrates an example user interface for guiding diagnosis or treatment of a medical condition.

FIG. 4 is block diagram showing generation of a data trace and associated treatment pathway for use in machine learning systems to effect diagnosis or treatment of a medical condition.

FIG. 5 is an example computing environment including a machine learning system to effect diagnosis or treatment of a medical condition.

FIG. 6 shows a flow diagram including an example process for guiding diagnosis or treatment of a medical condition.

FIG. 7 is a diagram of an example computing system.

Like reference numbers and designations in the various drawings indicate like elements.

### DETAILED DESCRIPTION

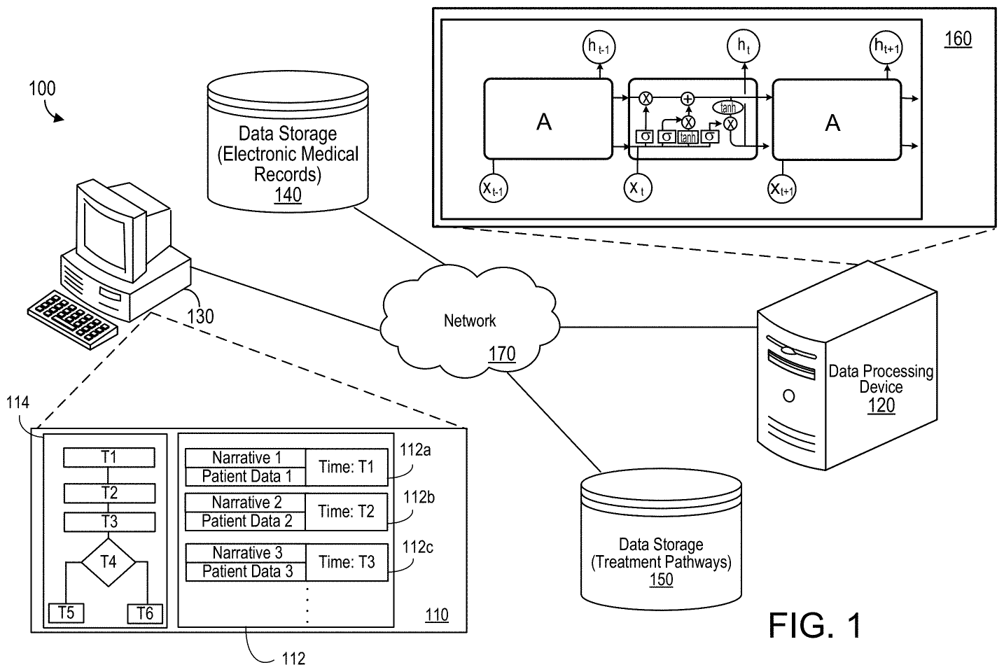

FIG. 1 shows an example of a data processing system 100 including clinical decision support tools to provide guidance for treatment or diagnosis of a medical condition. Generally, the clinical decision support tools include a treatment pathway interface 110 associated with the medical condition. Generally, data processing system 100 is configured to guide a user through a treatment or diagnosis process according to a treatment pathway 114, which can include a treatment guideline, a Care Pathway (CP), and so forth. The data processing system 100 includes a client device 130. The client device 130 is configured to host the interface 110 and present the interface to a user, such as a trained medical professional. The client device 130 is connected over a network 170 to a data processing device 120, a data storage 140 for storing Electronic Medical Records (EMR) including patient data, and a data storage 150 for storing treatment pathways (e.g., care pathways and guidelines), data traces, and the associations between and among data traces and treatment pathways. Generally, the data processing device 120 includes one or more machine learning engines 160 configured to receive patient data and data traces and recommend diagnoses or treatment for medical conditions of the patient.

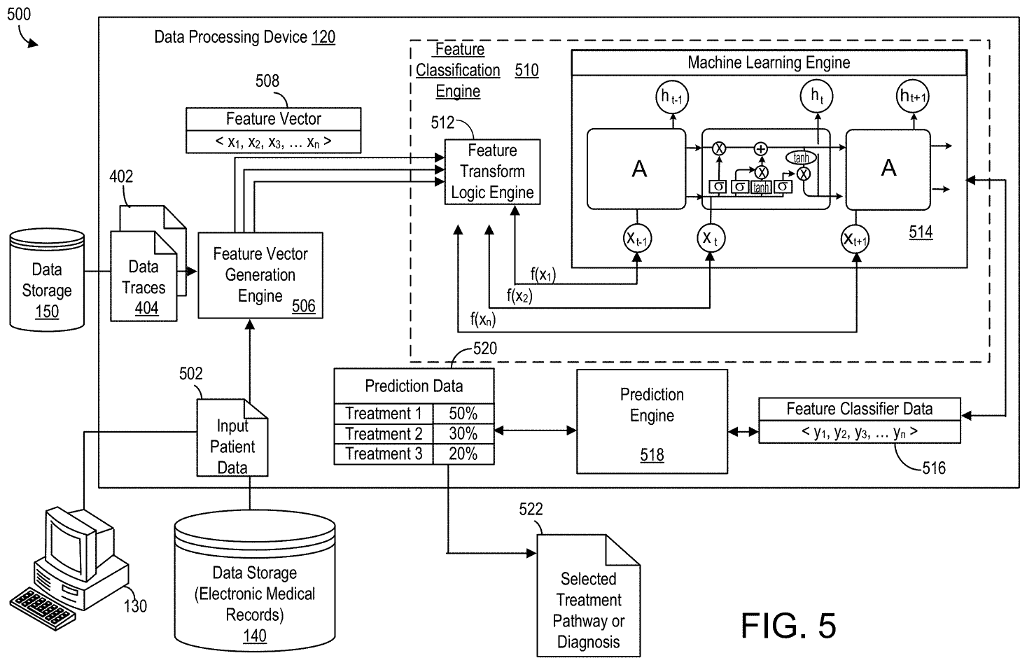

The treatment pathway 114 represents a standardized or semi-standardized process for diagnosing a medical condition in a patient or treatment of a medical condition of a patient. The interface 110 is configured to guide the user (e.g., a physician or other medical service provider) to treat a patient using the treatment pathway 114. In some implementations, the interface 110 assists a physician or other medical service provider for choosing the correct treatment pathway using machine learning processes, subsequently described in detail in relation to FIG. 5. The interface 110 ensures that each step of the treatment pathway is traversed during treatment. The interface 110 ensures that patient data that should be provided to the system for treatment or diagnosis of a medical condition is actually collected at a stage of the treatment pathway. Only when relevant data are collected, the interface 110 allows traversal of the treatment pathway to a subsequent step. Generally, the treatment pathway 114 can include one or more of several clinical decision support tools. These clinical support tools include a set of instruments used in healthcare that include guidelines and care pathways. For example, the interface 110 can present an interactive treatment pathway 114 for clinicians.

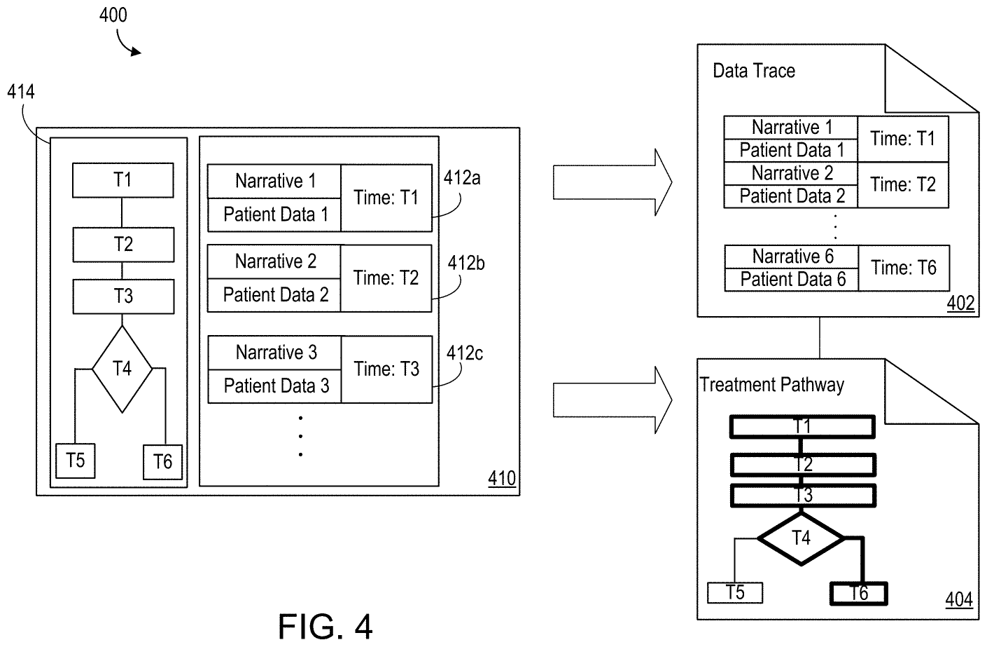

The data processing system 100 uses the data collected during the guided traversal of the treatment pathway 114 at each step to generate a data trace 112, described further in relation to FIG. 4. The data trace 112 represents how the treatment pathway was traversed and what patient data are associated with each stage of the treatment pathway. The data trace provides a standardized or semi-standardized data source that is compatible with one or more machine learning processes 160 to enable machine learning systems to provide guidance for treatment or diagnosis of medical conditions. The data processing system 100 uses these data traces and various machine learning processes 160 to suggest or effect a particular treatment or diagnose a particular medical condition for the patient.

To collect the data trace 112, the data processing system 100 uses the treatment interface 110. The interface 110 provides a step-by-step guidance through a treatment pathway. The interface 110 increases the granularity of data collected about what happened to a patient during a visit and it reduces clinician time to generate the clinical notes describing the patient encounter. Instead of simply referring to and following the presented procedures, the interface 110 allows clinicians to indicate where they are within a pathway and the branches they follow, and then automatically generate a detailed narrative description of what happened. At the end of an encounter with a patient, clinicians can scan over the generated narrative and make small changes to more accurately describe the encounter, such as documenting why and how they may have deviated from the treatment pathway 114.

The interface 110 generally presents the treatment pathway 114 and portions of the data trace 112 as the system traverses the treatment pathway. For example, the treatment pathway 114 includes steps T1, T2, T3, decision point T4, and alternate pathways T5 and T6. At step T1, the interface 110 prompts the clinician to collect patient data 1. The data processing system 100 may also auto-generate an associated narrative, narrative 1. A time stamp T1 is also included. This data together are a first element 112a of the data trace 112 being generated for the patient. Subsequent elements 112b and 112c are generated as the treatment pathway is traversed to T2, T3, and so forth.

The data trace 112 is generated by the data processing system 100 (e.g., by client device 130 or data processing device 120) in such a way so that the elements 112a, 112b, 112c can be transformed into features for use by a machine learning engine 160. Each element 112a-c of the data trace includes additional patient data that describes a patient status at each step of the guidance or care pathway. For example, the additional patient data can include any physiological value of the patient measured by the clinician during the traversal of the treatment pathway. These physiological parameters can include a heart rate, SpO₂ level, height, weight, body temperature, etc. of the patient. Patient data can also be descriptive, such as that the patient has a redness of complexion, is coughing, and so forth. These non-numerical data are standardized into the data trace by the interface 110. These can be used to auto-generate narrative data describing actions taken by the clinician and describing results received at each stage. Additional details can be added to the narrative data or the patient data by the clinician. However, the interface 110 generally requests that all data associated with a stage of the treatment pathway 114 be collected prior to traversing to the next stage.

Generally, the data storage 140 including the EMR and the data storage 150 including the treatment pathways are connected in the data processing system 100. These two data stores 140, 150 represent systems that are typically not connected to one another. The data processing system 100 connects these two systems for generating the data trace 112 for diagnosis and treatment of a medical condition of the patient. Generally, the EMR stores information about an individual patient's interaction with their healthcare provider. This dataset typically has clinical notes written by clinicians after each patient's interaction with a provider. When a patient visits a provider, clinicians access their records in the EMR and record new data there. The treatment pathway database 150 stores a list of care pathways and/or treatment guidance protocols. The treatment pathways, as previously stated, detail the actions a clinician should take in order to diagnose or treat a medical condition. For example, a care pathway can include a flow charts documenting the standard of care for a patient given particular patient data as an initial condition. For these pathways, there is a detailed and agreed upon standard of care. In some implementations, the data storage 140 and the data storage 150 can be stored in the same database or data storage.

In an example, when a patient arrives at a medical service provider (e.g., an emergency room or other treatment location), a medical service provider (e.g., a triage nurse) collects patient data about the patient and puts the patient data into a triage interface connected with the EMR. The data processing system 100 provides the triage information to the data processing device 120. The data processing device 120 can determine, from the patient data, which treatment pathway to retrieve from the data storage 150. This can be done using the machine learning engine 150, as described below. In an example, if the nurse input that the patient had a fever and is 4 months old, this information would go as a search data processing device 120. The data processing device 120 would access the data storage 150 including the library of treatment pathways and return the treatment pathway for treating an infant who presents with a fever. This reduces the amount of time a clinician must search for the data store 150 to treat the patient.

Additionally, physicians may be unaware of a treatment pathway's existence. For example, this is particularly true for medical residents in training or other clinicians who have little time to learn how information is organized within the institution. Clinicians would likely benefit greatly from the knowledge contained in care pathways. In thinking about patient data and the proper treatment pathway, physicians often rely initially on the triage keywords assigned to patients during their check-in. These keywords are shown during the patient overview screen of the EHR, so physicians know information about each patient and can prioritize who to see first. The interface 110 is configured to automatically suggest relevant care pathways by using this triage information to automate the search and return a ranked list of pathways that may be relevant to the current patient. By logging the triage keywords and what pathways are selected, the data processing system 100 improves over time by monitoring what pathways physicians select from the generated list. This is done by associating the chosen pathway with the patient data received for that pathway.

The data traces, treatment pathways (or identifiers representing them), and patient data associated with the treatment pathways and data traces can be inputs to the machine learning engine 160. For example, the data processing device 120 can be configured to generate a classifier for selecting the treatment pathway data for retrieval from the data storage. The classifier can be configured to determine a probability value associated with each treatment pathway of the treatment pathway data, the probability value indicative of a likelihood of the treatment pathway being responsive to the medical status represented in the patient data. The classifier can be used by the data processing device 120 to select, for retrieving from the data storage 150, the treatment pathway data comprising the treatment pathway based on the probability value associated with that treatment pathway. In some implementations, the classifier is generated using a machine learning process. For example, generating the classifier can include receiving training data comprising data traces for a plurality of patients, where each data trace being associated with a particular treatment pathway. The data processing device 120 trains a classification function of the machine learning engine 160 using the training data. The classification function is configured to determine the probability value associated with each treatment pathway of the treatment pathway data. Other similar examples for selecting treatment pathways ware possible.

The data processing system 100 is configured for enhanced presentation of clinical decision support systems (CDSS), including the treatment pathway 114. The interface 110 is configured to standardize the appearance of the treatment pathways 114 with a consistent layout and design.

While treating the patient using the interface 110, the clinician interacts with details of the treatment pathway 114 indicating what has been done (e.g., tests, labs, vitals, treatments). As the clinician takes these actions, the client device 130 (or other host of the interface 110) generates detailed clinician notes 112a-c, describing how the clinician is interacting with the patient. At the conclusion of the visit, the client device 130 inputs these automatically generated clinical notes into the EMR record 140 for the specific patient.

This reduces a documentation burden for clinicians because it improves the accessibility and utility of CDSS by applying principles of human-computer interaction. Rather than non-uniform, static files (e.g. PDF, Word, and Powerpoint files, etc.) or HTML pages, accessible only by searching on an intranet, the interface provides an adaptable, interactive resource. The treatment pathways 114 are interactive in several ways. First, pathways 114 will be automatically filtered to each physician's context. For example, for a pathway 114 deployed in an emergency room (ER), only steps relevant to the ER will be shown. Physicians will be able to “zoom out” to see the full pathway, if needed, but otherwise will not be distracted by steps not relevant to their current patient at the current point in time. Second, the interface 110 will support interaction allowing a clinician to indicate where their patient is on the pathway 114 and what was has been done. For example, physicians will be able to indicate if a step has been completed or choose a particular branch (e.g., T5 or T6) to indicate that portion of the pathway is relevant to their patient. Physicians will also be able to indicate if certain steps are not relevant and thus indicate a deviation. These interactions speed up the clinical note authoring process and generate the notes in such a way that a machine learning engine 160 can use the notes for applications such as selecting treatment pathways for similar patients and for updating how the system automatically generates notes for the pathway in the future. Based on the physicians' interactions with the care pathway, the interface 110 notes generator creates text (e.g., narrative 1, narrative 2, narrative 3, etc.) that the physicians can edit and input into their EHR note. The notes are interactive, so physicians can modify auto-generated content, adding more detail that was not present in the care pathway.

In a particular embodiment, the data processing system 100 uses the log of interactions to generate a draft of the notes. In some implementations, the data processing system can generate notes using natural language processing (NLP) to generate clinical notes based on our set of examples.

For example, a natural language processing (NLP) engine can be used to parse text from manually entered narrative data or patient data for subsequent auto-generation of narrative data and/or patient data. For example, NLP engines including topic models and neural networks with word embedding inputs can be used to assess the narratives and associate them with the treatment pathways to generate data.

In some implementations, the data processing device 120 is configured to map the topics and sentiments conveyed in natural language narrative entries to measures of the patient's medical condition using one or more natural language processing algorithmic approaches. Examples of NLP techniques include Latent Dirichlet Allocation, capturing the topics of the entry. The NLP techniques include positive and negative sentiment of the words used. The NLP techniques include deep neural network word embeddings. Other NLP techniques can also be applied to the narrative entries of a data trace. Each of those natural language models 330 outputs a score that is entered into a regularized logistic regression model using a LASSO (Least Absolute Shrinkage and Selection Operator) or other prediction method. In the LASSO example, cross validation is used to select natural language factors that best predict features of the narrative entries.

The data processing device 120 can semi-automatically generate clinical notes from the pathway 114. A variety of machine learning approaches to optimize the note generation. For example, a rule-based NLP system can be used based on two observations: 1) it is difficult to access a large corpus of clinical notes associated with CDSS, to train a more complex language model, since there is currently no explicit linkage between the notes and pathways 114, and 2) the current practice of documentation is suboptimal.

The data processing device 102 can be improved based on feedback from users. For example, clinicians can comment on the presence or absence of certain terms, and if they believe those terms should be present. By iterating on this process, the data processing system 100 identifies the specific information need from clinicians to enter as they interact with the interface 110, in order to produce a robust draft of the clinical notes. In some implementations, the data processing system 100 identifies specific sections of the generated notes to highlight for clinicians to manually add details to improve subsequent generation of notes.

The data processing system 100 maps the data trace 112 from the CDSS into the standardized nomenclature used in the EHRs, including order sets, ICD-10 codes, and CPT codes. This representation will maintain all of the states, sequences, and references currently present in CDSS pathways. The data processing system 100 can analyze the clinical notes, looking for connections between the structured data in the CDSS and the unstructured information that was recorded in the clinical notes. Notes can be used from many clinicians over many instances of the data processing system 100 in order to understand different writing styles and generate a robust base of data for training the machine learning engine 160. Terms can be weighted based on feedback provided to the system, such as what clinicians find most useful in terms of note content and how parts of the pathway 114 might map to specific elements of the final clinical notes. The data processing device 120 can be configured to author individual sentences for each action that can be taken within a pathway 114 (e.g., CDSS).

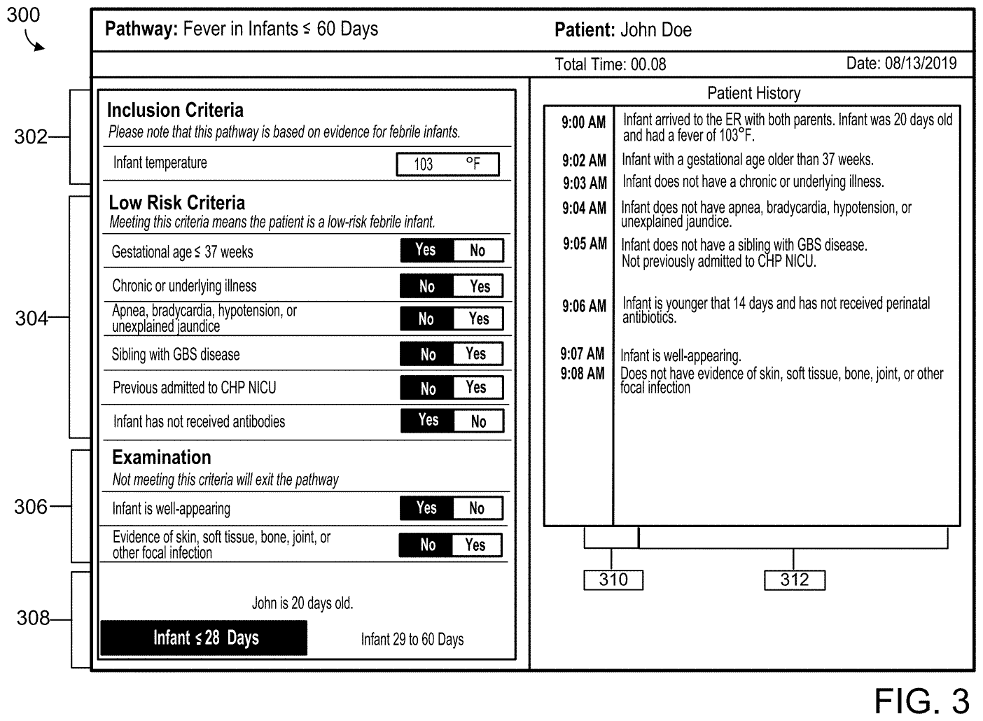

FIG. 3 shows We demonstrate an example of our interactive CDSS prototype in FIG. 3, utilizing the structure of ‘Fever in Infants under 60 days’ care pathway from Children's Hospital of Pittsburgh. In this example, a 20-day old child visits the ER with both parents, with a temperature of 103 degrees. The child met all the criteria to be considered a low-risk febrile infant, given his lab results of ANC 3800/mm, PCT 1.5 ng/ml, UA with 9 WBC/mm, and negative CXR. Based on the recommendations of the pathway, the physician agreed to not administer antibiotics and the child was discharged at the end of the visit. After discharge, the physician used the semi-automated clinical notes to speed up the documentation process and moved on to treat the next patient. Furthermore, our CDSS would log time-stamped, low-level data, including all of the decision points for being considered low-risk. This type of low-level information is often not automatically kept in a structured format in EHRs. After increasing volumes of patient encounters are documented using our interactive CDSS, healthcare institutions will be able to accurately assess if there is compliance with each care pathway, and if its use has led to successful health outcomes for patients by analyzing the rich log data, serving as a critical step in bringing data-driven innovation to healthcare.

In some implementations, the detection device 110, data processing device 120, and client device 140 are included in a single computing system, and the functions of these devices can be executed using one or more processors of that computing system.

Generally, the interface 110 can use real-time data collection. The patient data can be sent to the data processing device 120 for combining with statistical machine learning algorithms, to retrieve the proper treatment pathway data 114. The interface 110 is configured to collect input data such as, baseline demographic information, pregnancy history (e.g., miscarriage, prior preterm birth), conception method (e.g., natural, IVF, ovulation drugs), medical history (e.g., diabetes, hypertension), behavior (e.g., drugs/tobacco/alcohol), patient physiology, and other patient data. Once the patient data are received by the interface 110, the patient data can be sent to the data processing device 120 for conversion into feature data as described in relation to FIG. 5. The feature data are classified by a feature classification engine of the data processing device 120. The feature classification engine is configured to classify the features as corresponding to a medical condition or diagnosis and suggest a treatment pathway for use for the patient. The data processing device 120 can store the results of the classification in a profile associated with the patient, such as in the EMR data storage 140 associated with the data processing device 120.

The results of the classification can be used for a variety of applications, such as facilitating remediation of the medical condition. Depending on which health conditions identified, the data processing device 120 (or other device of the computing environment 100) can help the physician or other user remediate the condition. In some implementations, the patient can be presented with a tentative diagnosis to be verified through the interface 110 in collaboration with the clinician (e.g., a physician). In some implementations, a summary of the diagnosis or medical condition can be generated and stored on the client device 130 and presented to a medical service provider at a later time.

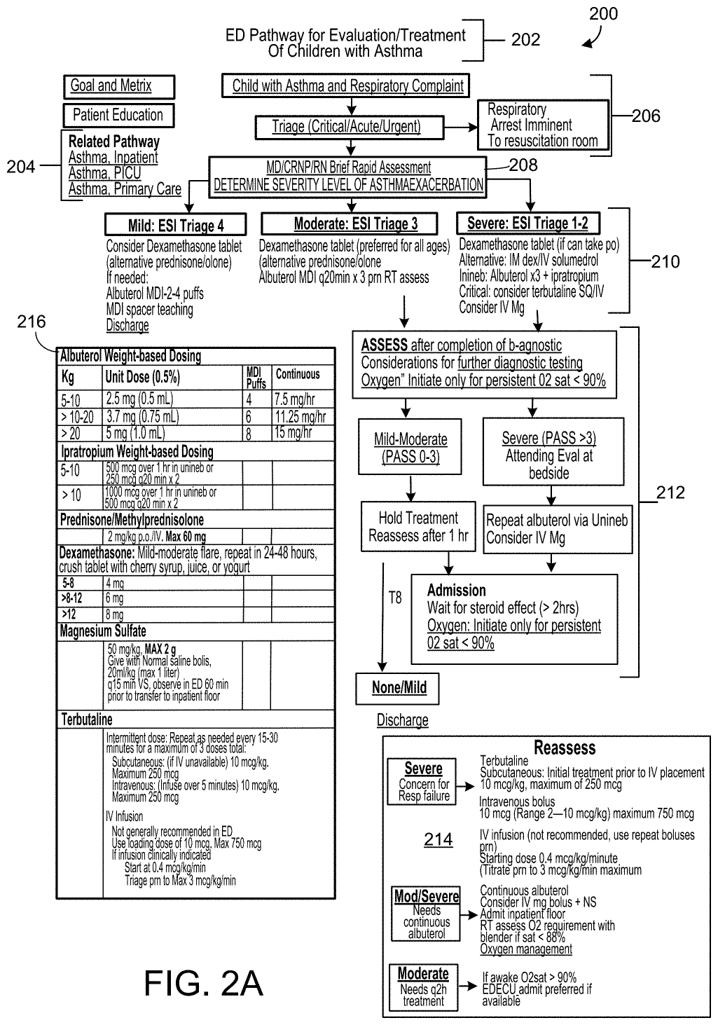

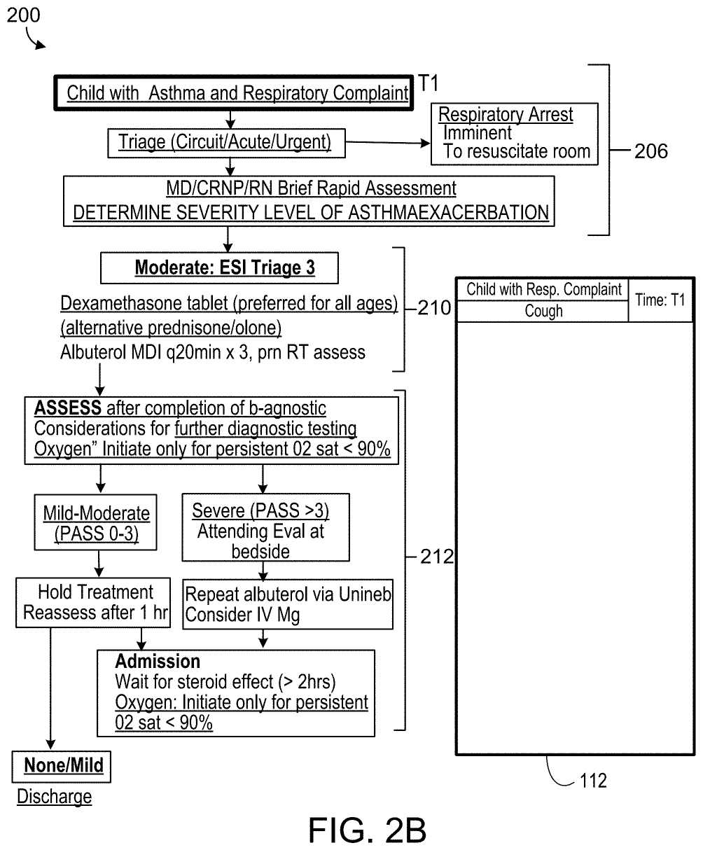

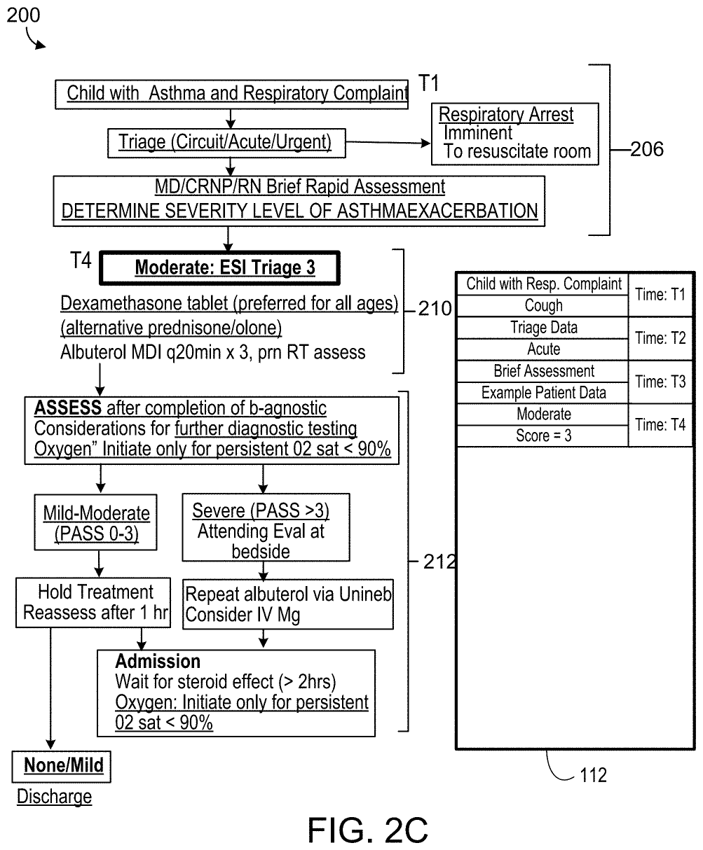

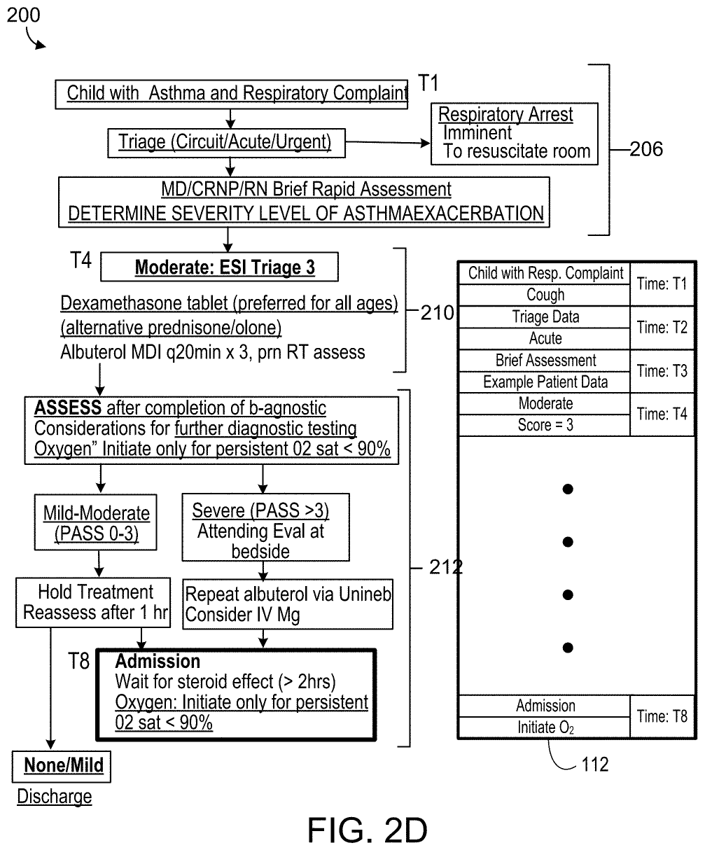

Turning to FIGS. 2A-2D, an example of a treatment pathway 200 is shown. In FIG. 2A, the pathway 200 is a care pathway for evaluation and/or treatment of a child with asthma. The pathway is associated with an identifier 202. The identifier 202 can include terms which assist the data processing device 120 in selecting the pathway based on patient data received via the interface 110 of FIG. 1. In some implementations, similar or related pathways 204 can be suggested by the data processing device 120 so that a clinician can select a different (but similar) pathway as needed. If an alternative pathway is selected that is not the suggested pathway by the data processing system 100, the data processing system logs this selection and uses it to update the classifier used to select the treatment pathway.

The pathway 200 is suggested based on initial patient data received by the data processing system 100. For example, a triage nurse collects patient data on the reason for the patient's arrival at the emergency department as well as information such as the patient's age. A nurse can enter “7-year-old girl with asthma, distressed breathing” into the triage system. This entry triggers a search for a relevant pathway (e.g., a CDSS) matching the current situation, using the classifier. When the physician enters the patient's assigned electronic medical record chart, they see a ranked list of possible pathway options and select “Child with Asthma” from the list (e.g., from the top of the list). In some implementations, the selection is performed automatically.

The pathway 200 includes a series of nodes 206, 208, 210, and 212 that are linked together to form the pathway. The interface 110 is configured to traverse the pathway by iterating through each node based on patient data received at a given node. For example, at nodes 206, a triage is performed to categorize the severity of the potential medical condition of the patient. Each of the nodes are associated with a suggested action or a request for data from the data processing system 100. When the action is taken and/or the additional patient data are received, the interface progresses to the next node. In some implementations, a single node is shown on the screen. In some implementations, the entire pathway 200 is shown to contextualize the current node, which can be emphasized (e.g., highlighted, etc.) on the screen.

As more patient data are acquired, the data processing system auto-generates clinical notes, which can appear on the screen (e.g., as shown in FIG. 3). Briefly turning to FIG. 3, in this example, the physician looks at the inclusion and exclusion criteria 302 and 304, the first set of elements for this care pathway. He confirms that the girl has previously been diagnosed with asthma, an important inclusion criteria. When he selects this item on the pathway 200, new text appears in the clinical notes 312 in the display with a new timestamp 310. The inclusion and exclusion criteria scroll up, making space for collection of data 306 and 308 that calculates an Asthma Distress Score (ADS). The physician observes the patient's breathing and inputs 27 (breaths per minute) into the first text box on the pathway 200. In reaction, the ADS panel highlights 25-30 bps and displays a score of 1 at the bottom of the panel. In addition, the clinical notes on the right show a new entry at 7:44 with the patient's breathing rate. Next, the physician uses a stethoscope and listens to the patient's breathing. They interact with the ADS panel, selecting “decreased at the bases” for air entry and selecting “nasal flaring” for work of breathing. This updates the ADS score to 3, and it automatically adds additional data to the clinical note.

Returning to FIG. 2A, the “moderate” branch of the pathway 200 is selected at decision point 208. This is because of the ADS score being in the range suggesting moderate severity. Each of the branches 210 can be selected based on the patient data at the decision point 208. In this case, the traversal processes to the branches of nodes 212 for moderate severity. Once each node is traversed and relevant patient data obtained, the clinician can reassess the patient and take an action as indicated by a final reassess node 214.

In some implementations, additional contextual information 216 can be presented along with the pathway 200. Here, dosages are shown for different patient weights. This display can be dynamic, such that the displayed information 216 can updated based on which node is currently being traversed on the pathway 200.

Turning to FIGS. 2B-2D, examples of the interface are shown as the user progresses through the pathway. In FIG. 2B, the interface 110 shows the first node at time T1, at the beginning of the pathway 200. Some initial patient data has been included in the data trace 112 associated with the pathway 200. As the user progresses through the pathway 200, the data trace 112 is updated, as shown in FIG. 2C. At time T4, at the fourth node in the pathway 200, the data trace 112 has been populated with four entries of patient data corresponding to the nodes of the pathway 200. In FIG. 2D, the end of the pathway 200 is reached, progressing through the moderate branch. At time T8, patient data indicate that the patient should be admitted. This is added to the data trace 112. At the conclusion of traversing the pathway 200, further actions can be suggested as previously described. The populated data trace 112 can be stored in the patients EMR. In some implementations, the data trace 112 can be used for updating the classifiers of the machine learning model, as subsequently described.

Returning to FIG. 3, once the pathway 200 is completed, a data trace including the narratives 312, timestamps 310, and patient data collected during the traversal is generated. The data trace automatically includes high-quality labels for the data which facilitate classification of a medical condition or diagnosis for the patient.

In some implementations, the pathway 200 can be refined based on data traces associated with that pathway. For example, if a large percentage of clinicians deviate from the pathway 200 at a given decision point, or deviate from a suggested branch or use different data thresholds for doing so than recommended, the pathway can be updated to reflect this consensus. For example, if a pathway suggests branch A when a value is below 100, but clinicians routinely select branch A when the value is below 120, the threshold value may be changed to 120. This may be otherwise flagged for assessment prior to automatically refining the pathway 200.

Turning to FIG. 4, a process 400 for generating the data trace 402 is shown. As previously stated, the interface logs time-stamped, low-level data, such as the patient's actual breaths per minute, a description of the air entry, and a description of the patient's effort to breathe to generate the data trace. Generally, current CDSS do not log this information. At most, the patient's current standard EHR captures that the patient has an ADS score of three, but there would be no detail of which measures produced that score. Hours later, the physician would sit down at a computer and work to generate a narrative account of what happened based on their recollection and on the sparse details entered into the EHR. The interface generates richer trace data that are used to iteratively improve the quality of the Asthma pathway 200, to assess the speed and quality of new procedures or organizational structures in the emergency department, to aid machine learning systems in learning which features matter most when modeling a more personalized set of diagnosis or treatment alternatives, and to help clinicians more efficiently author more accurate and detailed clinical notes. The data processing system's 100 first draft of the clinical notes reduces the amount of time clinicians spend writing up their notes by providing them with the details of what transpired.

Here, a pathway 414 is associated with narrative data, patient data, and time stamp data entries 412a, 412b, and 412c. Once the pathway 412 is traversed, the entries 412a-c are combined into a trace 402. This data trace 402 is linked to the particular treatment pathway 404 traversed. Here, the pathway is shown as T1, T2, T3, T4, and T6, skipping the branch including T5. The data trace can be sent to a machine learning engine for use in refinement of the treatment pathway 414, generation of a classifier for selecting the treatment pathway 414 for given patient data, and selection of a diagnosis or treatment for a medical condition for effecting the diagnosis or treatment.

FIG. 5 shows an example of a data processing system 500. The data processing system 500 in this example includes the data processing device 120 of FIG. 1. The data processing system 500 shows the client device 130 and the data processing device 120 as different computing devices, but the devices can be combined into a single computing device. The data processing device 120 includes feature vector generation engine 506, a classification engine 510 and a prediction engine 518. The feature classification engine 510 and the prediction engine 518 are in communication with each other and with the client device 130.

The client device 130 is configured to display interface 110 with which the clinician (or other user) can interact. Patient data 502 are provided from the interface and from EMR 140. The data processing device 120 processes these inputs to determine features (e.g., parameters) that are indicative of the user's medical condition and for which treatment pathway should be selected, depending on the classification being performed.

The input data 502 are transformed into features of a feature vector 508 by feature vector generation engine 210 (such as using the NLP models described in relation to FIG. 1). The feature vector 508 concisely represents the characteristics of the patient data for the patient and/or characteristics of pathways from the data storage 150. For example, the feature vector can be generated by the feature vector generation engine based on parsing the text of the narratives associated with the pathways and comparing discovered words in the text to items in one or more data dictionaries. A data dictionary can specify words or phrases that correspond to features for including in the feature vector 508.

The feature vector generation engine 506 generates a high-dimensional vector including one or more features that are extracted from the input patient data 502 and/or from the data traces 404 and associated pathways 402. In some implementations, the features can correspond to the words or phrases of the data dictionary.

The feature vector 508 is sent from the feature vector generation engine 506 to the feature classification engine 510. The feature classification engine 510 includes logic that transforms the feature vector 508 into data that can be processed by machine learning logic 514. The feature classification engine includes a feature transform logic engine 512 and machine learning engine 514 (e.g., engine 160 of FIG. 1).

The feature transform logic engine 512 transforms the feature vector 508 into inputs for the machine learning engine 514. For example, the feature transform logic 512 can normalize the features of the feature vector 508 to values that can be recognized by the machine learning logic 514. For example, the feature vector 508 can be transformed into activation inputs for a neural network. In some implementations, the machine learning engine 514 includes a support vector machine. In some implementations, the machine learning engine 514 includes a convolutional neural network (CNN). In some implementations, the features of the feature vector are transformed into values between 0 and 1 through a non-linear transformation, where the normalized value represents an activation level for the neural network, and where the normalized scale is a non-linear representation of the values of the features before the normalization process. The values to which the features are transformed can depend on a type of machine learning engine 514 being used, and the weighting scheme associated with the machine learning engine 514.

The machine learning engine 250 is configured to receive the normalized features of the feature vector 508 and computes classification data 516, such as through a deep learning process. For example, neural network logic can include a long short-term memory (LSTM) neural network, which tracks dependencies between features of the feature vector 508. Other recurrent neural networks can be used. Other machine learning classifiers can be used as well.

The feature classifier data 516 includes one or more output values <y₁ . . . yₙ> of the machine learning engine 514. For example, each output can be a classification value for one or more features of the feature vector 508. Each value of the classifier data 516 can indicate whether medical condition is represented or not represented in the features of the input patient data, data trace, etc. for diagnosing or treating the patient and/or for selecting a pathway for that purpose.

The classifier data 516 is sent to a prediction engine 518. The prediction engine 518 is configured to assign probabilities to one or medical conditions as being present for the patient. The prediction data 520 shows the likelihood that each of one or more medical conditions is present for the patient and a treatment that should be recommended. The collection of health risks and their associated probabilities of the probabilities data 520 can together be used to determine if the patient has a disease or other health condition. For example, if a user is showing health risks including high anxiety, high apathy, etc., a health condition of depression can be identified for that patient. The probabilities data 520 can be presented to the patient or used to trigger a remediation action, such as selection of a pathway 522.

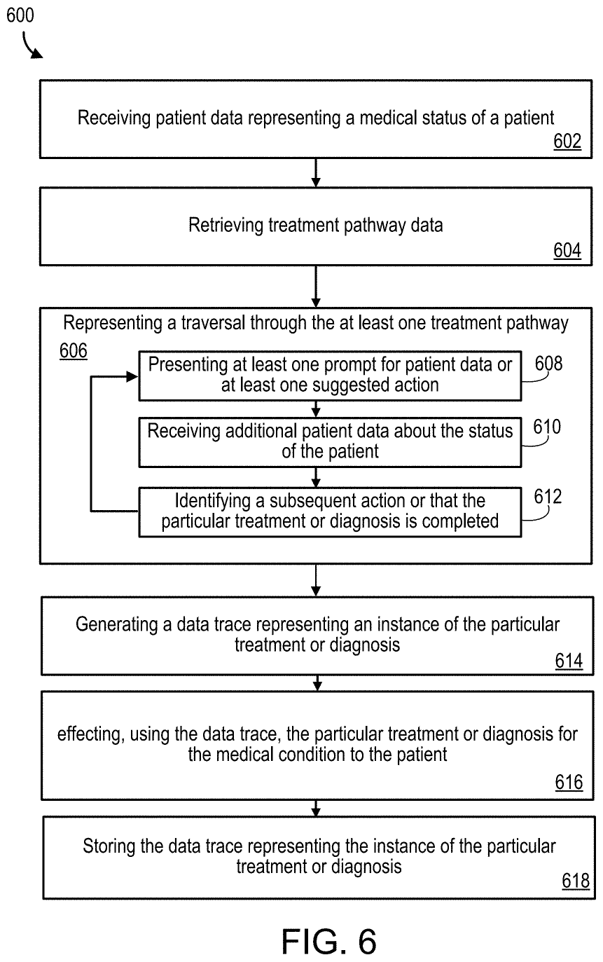

FIG. 6 shows an example process 600 for treating a patient for a medical condition using an interactive treatment pathway interface associated with the medical condition. Generally, the process 600 is executed by the data processing system 100. The process 600 includes receiving (602), through an interface (e.g., interface 110), patient data representing a medical status of a patient. The process 600 includes retrieving (604), from a data storage (e.g., data store 150) based on the patient data, treatment guidance data comprising a plurality of nodes, the plurality of nodes being linked to one another to form at least one treatment pathway through at least a portion of the plurality of nodes, the at least one treatment pathway representing a sequence of actions for a particular treatment or diagnosis of a medical condition of the patient. Selection and retrieval of the treatment guidance data can be performed using a machine learning process. For example, the data processing system can be configured to generate a classifier for selecting the treatment guidance data for retrieval from the data storage. The classifier can be configured to determine a probability value associated with each treatment pathway of the treatment guidance data, the probability value indicative of a likelihood of the treatment pathway being responsive to the medical status represented in the patient data. Using the classifier, the data processing system is configured to select, for retrieving from the data storage, the treatment guidance data comprising the treatment pathway based on the probability value associated with that treatment pathway.

In some implementations, the process includes generation of the classifier using patient data and data traces. Generating the classifier can include receiving training data comprising data traces for a plurality of patients, each data trace being associated with a particular treatment pathway. The data processing system can be configured for training a classification function using the training data, the classification function configured to determine the probability value associated with each treatment pathway of the treatment guidance data.

The process 600 includes representing (606), by the interface, a traversal through at least the portion of the nodes representing the at least one treatment pathway to guide the particular treatment or diagnosis of the medical condition, the traversal comprising, for each node represented in the at least one treatment pathway. Generally, the traversal includes presenting (608), by the interface, at least one prompt for patient data or at least one suggested action for the particular treatment or diagnosis of the medical condition. In some implementations, the interface is configured for presenting, to a user of the interface, a representation of the particular treatment or diagnosis, represented in the data trace. Generally, the traversal includes receiving (610), in response to presenting the at least one prompt, additional patient data about the status of the patient. Generally, the traversal includes identifying (612), based on the additional patient data, either a subsequent node in the at least one treatment pathway or that the particular treatment or diagnosis is completed.

The process 600 includes generating (614), once traversal of the pathway is completed, from the additional patient data received during the traversal, a data trace representing an instance of the particular treatment or diagnosis of the medical condition of the patient. The process 600 includes effecting (616), using the data trace, the particular treatment or diagnosis for the medical condition to the patient. The process 600 includes storing (618) the data trace representing the instance of the particular treatment or diagnosis in the data storage.

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, the data processing device 120, the client device 130, and the data storage 140, 150 can each be implemented using digital electronic circuitry, or in computer software, firmware, or hardware, or in combinations of one or more of them. In another example, the processes 500 and 600, can be implemented using digital electronic circuitry, or in computer software, firmware, or hardware, or in combinations of one or more of them.

Some implementations described in this specification (e.g., the machine learning engine 160, etc.) can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. In some implementations, the client device 130 and/or the data processing device 120 include a data processing apparatus as described herein. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed for execution on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD-ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

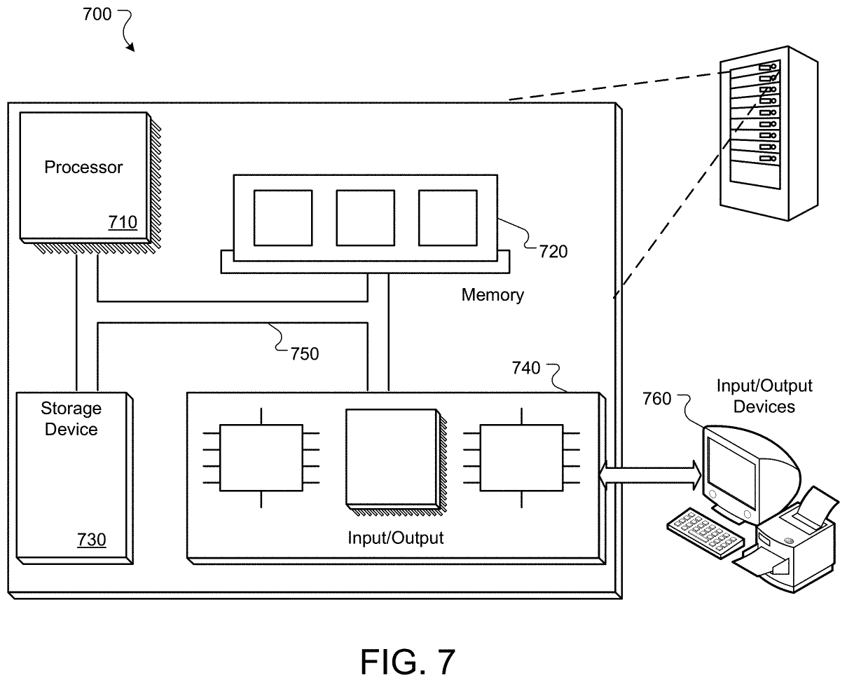

FIG. 7 shows an example computer system 700 that includes a processor 710, a memory 720, a storage device 730 and an input/output device 740. Each of the components 710, 720, 730 and 740 can be interconnected, for example, by a system bus 750. The processor 710 is capable of processing instructions for execution within the system 700. In some implementations, the processor 710 is a single-threaded processor, a multi-threaded processor, or another type of processor. The processor 710 is capable of processing instructions stored in the memory 720 or on the storage device 730. The memory 720 and the storage device 730 can store information within the system 700.

The input/output device 740 provides input/output operations for the system 700. In some implementations, the input/output device 740 can include one or more of a network interface device, e.g., an Ethernet card, a serial communication device, e.g., an RS-232 port, and/or a wireless interface device, e.g., an 802.11 card, a 3G wireless modem, a 4G wireless modem, a 5G wireless modem, etc. In some implementations, the input/output device can include driver devices configured to receive input data and send output data to other input/output devices, e.g., keyboard, printer and display devices 760. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of embodiments have been described. Nevertheless, it will be understood that various modifications may be made without departing from the spirit and scope of the data processing system described herein. Accordingly, other embodiments are within the scope of the following claims.

What is claimed is:

1. A method for treating a patient for a medical condition using an interactive treatment pathway interface associated with the medical condition, the method comprising:
receiving, through an interface, patient data representing a medical status of a patient;
retrieving, from a data storage based on the patient data, treatment guidance data comprising a plurality of nodes, the plurality of nodes being linked to one another to form at least one treatment pathway through at least a portion of the plurality of nodes, the at least one treatment pathway representing a sequence of actions for a particular treatment or diagnosis of a medical condition of the patient;
representing, by the interface, a traversal through at least the portion of the nodes representing the at least one treatment pathway to guide the particular treatment or diagnosis of the medical condition, the traversal comprising, for each node represented in the at least one treatment pathway:
presenting, by the interface, at least one prompt for patient data or at least one suggested action for the particular treatment or diagnosis of the medical condition;
receiving, in response to presenting the at least one prompt, additional patient data about the status of the patient; and
identifying, based on the additional patient data, either a subsequent node in the at least one treatment pathway or that the particular treatment or diagnosis is completed;
generating, from the additional patient data received during the traversal, a data trace representing an instance of the particular treatment or diagnosis of the medical condition of the patient;
effecting, using the data trace, the particular treatment or diagnosis for the medical condition to the patient; and
storing the data trace representing the instance of the particular treatment or diagnosis in the data storage.

2. The method of claim 1, further comprising:
presenting, to a user of the interface, a representation of the particular treatment or diagnosis, represented in the data trace.

3. The method of claim 2, wherein the user of the interface is a medical professional.

4. The method of claim 1, further comprising:
generating a classifier for selecting the treatment guidance data for retrieval from the data storage, the classifier being configured to:
determine a probability value associated with each treatment pathway of the treatment guidance data, the probability value indicative of a likelihood of the treatment pathway being responsive to the medical status represented in the patient data; and
select, for retrieving from the data storage, the treatment guidance data comprising the treatment pathway based on the probability value associated with that treatment pathway.

5. The method of claim 4, wherein the classifier is generated using a machine learning process.

6. The method of claim 4, wherein generating the classifier comprises:
receiving training data comprising data traces for a plurality of patients, each data trace being associated with a particular treatment pathway;
training a classification function using the training data, the classification function configured to determine the probability value associated with each treatment pathway of the treatment guidance data.

7. The method of claim 1, further comprising:
generating, in the data trace for each node of the treatment pathway, description data comprising a natural language description of the additional patient data associated with the node.

8. The method of claim 7, wherein the natural language description is generated using a machine learning process.

9. The method of claim 1, wherein the data trace comprises, for each node traversed, a time stamp representing when an action for treatment of the medical condition of the patient is performed.

10. A system for treating a patient for a medical condition using an interactive treatment pathway interface associated with the medical condition:
a data storage for storing one or more instructions;
one or more processing devices in communication with the data storage and configured to execute the one or more instructions to perform operations comprising:
receiving, through an interface, patient data representing a medical status of a patient;
retrieving, from a data storage based on the patient data, treatment guidance data comprising a plurality of nodes, the plurality of nodes being linked to one another to form at least one treatment pathway through at least a portion of the plurality of nodes, the at least one treatment pathway representing a sequence of actions for a particular treatment or diagnosis of a medical condition of the patient;
representing, by the interface, a traversal through at least the portion of the nodes representing the at least one treatment pathway to guide the particular treatment or diagnosis of the medical condition, the traversal comprising, for each node represented in the at least one treatment pathway:
presenting, by the interface, at least one prompt for patient data or at least one suggested action for the particular treatment or diagnosis of the medical condition;
receiving, in response to presenting the at least one prompt, additional patient data about the status of the patient; and
identifying, based on the additional patient data, either a subsequent node in the at least one treatment pathway or that the particular treatment or diagnosis is completed;
generating, from the additional patient data received during the traversal, a data trace representing an instance of the particular treatment or diagnosis of the medical condition of the patient;
effecting, using the data trace, the particular treatment or diagnosis for the medical condition to the patient; and
storing the data trace representing the instance of the particular treatment or diagnosis in the data storage.

11. The system of claim 10, wherein the operations further comprise:
presenting, to a user of the interface, a representation of the particular treatment or diagnosis, represented in the data trace.

12. The system of claim 10, wherein the operations further comprise:
generating a classifier for selecting the treatment guidance data for retrieval from the data storage, the classifier being configured to:
determine a probability value associated with each treatment pathway of the treatment guidance data, the probability value indicative of a likelihood of the treatment pathway being responsive to the medical status represented in the patient data; and
select, for retrieving from the data storage, the treatment guidance data comprising the treatment pathway based on the probability value associated with that treatment pathway.

13. The system of claim 12, wherein the classifier is generated using a machine learning process.

14. The system of claim 12, wherein generating the classifier comprises:
receiving training data comprising data traces for a plurality of patients, each data trace being associated with a particular treatment pathway;
training a classification function using the training data, the classification function configured to determine the probability value associated with each treatment pathway of the treatment guidance data.

15. The system of claim 10, wherein the operations further comprise:
generating, in the data trace for each node of the treatment pathway, description data comprising a natural language description of the additional patient data associated with the node.

16. The system of claim 10, wherein the data trace comprises, for each node traversed, a time stamp representing when an action for treatment of the medical condition of the patient is performed.

17. One or more non-transitory computer readable media storing instructions that are executable by one or more processors configured to perform operations comprising:
receiving, through an interface, patient data representing a medical status of a patient;
retrieving, from a data storage based on the patient data, treatment guidance data comprising a plurality of nodes, the plurality of nodes being linked to one another to form at least one treatment pathway through at least a portion of the plurality of nodes, the at least one treatment pathway representing a sequence of actions for a particular treatment or diagnosis of a medical condition of the patient;
representing, by the interface, a traversal through at least the portion of the nodes representing the at least one treatment pathway to guide the particular treatment or diagnosis of the medical condition, the traversal comprising, for each node represented in the at least one treatment pathway:
presenting, by the interface, at least one prompt for patient data or at least one suggested action for the particular treatment or diagnosis of the medical condition;
receiving, in response to presenting the at least one prompt, additional patient data about the status of the patient; and
identifying, based on the additional patient data, either a subsequent node in the at least one treatment pathway or that the particular treatment or diagnosis is completed;
generating, from the additional patient data received during the traversal, a data trace representing an instance of the particular treatment or diagnosis of the medical condition of the patient;
effecting, using the data trace, the particular treatment or diagnosis for the medical condition to the patient; and
storing the data trace representing the instance of the particular treatment or diagnosis in the data storage.

18. The one or more non-transitory computer readable media of claim 17, wherein the operations further comprise:
generating a classifier for selecting the treatment guidance data for retrieval from the data storage, the classifier being configured to:
determine a probability value associated with each treatment pathway of the treatment guidance data, the probability value indicative of a likelihood of the treatment pathway being responsive to the medical status represented in the patient data; and
select, for retrieving from the data storage, the treatment guidance data comprising the treatment pathway based on the probability value associated with that treatment pathway.

19. The one or more non-transitory computer readable media of claim 18, wherein the classifier is generated using a machine learning process.

20. The one or more non-transitory computer readable media of claim 18, wherein generating the classifier comprises:
receiving training data comprising data traces for a plurality of patients, each data trace being associated with a particular treatment pathway;
training a classification function using the training data, the classification function configured to determine the probability value associated with each treatment pathway of the treatment guidance data.
